(19) United States
(12) Patent Application Publication
(10) Pub. No.: US 2013/0035640 A1
(43) Pub. Date: Feb. 7, 2013

(54) FLUID DRUG DELIVERY SYSTEM AND METHOD FOR MANUFACTURING A DRUG DELIVERY SYSTEM

(71) Applicants: Nils Basso, Frankfurt am Main (DE); Thomas Nagel, Tharandt (DE); René Richter, Frankfurt am Main (DE); Robert Witt, Frankfurt am Main (DE); Richard Guenther, Dresden (DE)

(72) Inventors: Nils Basso, Frankfurt am Main (DE); Thomas Nagel, Tharandt (DE); René Richter, Frankfurt am Main (DE); Robert Witt, Frankfurt am Main (DE); Richard Guenther, Dresden (DE)

(73) Assignee: SANOFI-AVENTIS DEUTSCHLAND GMBH, FRANKFURT AM MAIN (DE)

(21) Appl. No.: 13/319,313

(22) PCT Filed: May 10, 2010
(86) PCT No.: PCT/EP10/56352

§ 371 Date: Apr. 17, 2012

(30) Foreign Application Priority Data
May 13, 2009 (EP) 09006450.2

(51) Int. Cl. A61M 5/31

(52) U.S. Cl. 604/181

(57) ABSTRACT

A fluid drug delivery system comprises a rigid shell having an inner sidewall, an opening and a passage as well as a collapsible cartridge with a distal end and a proximal end. The collapsible cartridge is arranged within the shell with its distal end being closer to the opening than the proximal end. It further comprises a first portion including the proximal end and a second portion including the distal end. The second portion is recoilable from the inner sidewall to urge a fluid, which comprises a drug and is contained in the collapsible cartridge, to the passage of the rigid shell. Further, the rigid shell and the collapsible cartridge are co-extruded.

### CROSS REFERENCE TO RELATED APPLICATIONS

The present application is a U.S. National Phase Application pursuant to 35 U.S.C. §371 of International Application No. PCT/EP2010/056352 filed May 10, 2010, which claims priority to European Patent Application No. 09006450.2 filed on May 13, 2009. The entire disclosure contents of these applications are herewith incorporated by reference into the present application.

### FIELD OF INVENTION

The present invention is related to a fluid drug delivery system. The present invention also refers to a method for manufacturing such drug delivery system.

### BACKGROUND

Drug delivery systems or drug delivery devices are generally known for administration of a medicinal product, for example insulin, growth hormones or other drugs, being suitable for self-administration by a patient.

For this purpose, a drug delivery system may comprise a barrel or chamber into which a fluid to be injected is charged. A patient will then dispense the fluid comprising the drug into his body.

Some of those drug delivery devices are configured to deliver a plurality of different selectable doses. For that purpose, the drug delivery device may include a drive mechanism suitable for use in pen-type injectors, where an amount of pre-set doses of a medicinal product can be administered. The patient may attach a needle unit to the drug delivery device for dispensing the medicinal product into his body. After usage of the drug delivery device, the needle unit can be replaced. In addition, it may be useful to reuse the pen-type injector system and to replace only the cartridge including the fluid and the drug. This may allow a patient to become more familiar with his drug delivery device for a smoother and more convenient administration of drugs.

U.S. Pat. No. 6,620,134 shows a drug delivery device for such purpose including an elongated shell, a pressure member slidably disposed within the elongated shell, and a collapsible cartridge including the fluid to be delivered. The collapsible cartridge with the pre-filled injection fluid is loaded into the elongated shell. A piston rod of the dose dial system is connected to the pressure member slidably disposed within the elongated shell to provide the respective fluid by advancing the pressure member along the shell, thereby pressurizing the fluid within the collapsible cartridge through a needle unit connected to the drug delivery device.

However, loading of the collapsible cartridge into the elongated shell may be a complex and time-consuming procedure, even if the collapsible cartridge contains no fluid and the fluid is inserted afterwards. Furthermore, a collapsible cartridge with a pre-filled injection fluid may be damaged upon loading into the elongated shell or not fit smoothly enough to ensure the delivery of correct doses.

Consequently, there is still a need to provide a simple to use drug delivery device which overcomes the disadvantages of known devices.

### SUMMARY

For this object, a fluid drug delivery system may comprise a rigid elongated shell having an inner sidewall, an opening, and a passage. A collapsible cartridge with a distal and a proximal end is arranged within the elongated shell with its distal end being closer to the opening than the proximal end. The collapsible cartridge also comprises a first portion including the proximal end and a second portion including the distal end. The first portion is coupled to a passage of the rigid elongated shell and the second portion is in contact with the inner sidewall of the elongated rigid shell. The second portion is further recoilable from the inner sidewall to urge a fluid, which comprises a drug and is contained in the collapsible cartridge, through the passage. The rigid elongated shell and the collapsible cartridge comprise approximately the same length.

The rigid elongated shell and the collapsible cartridge may be co-extruded. The co-extrusion of the rigid-elongated shell and the collapsible cartridge results in a smooth and fitting contact of the second portion of the collapsible cartridge with the inner sidewall. No twists of the cartridge material, strain or stress may occur, which could weaken the collapsible cartridge or constrain the correct amount of injection fluid to be delivered.

In a further embodiment, a pressure member may be provided, which is adapted to be slidably disposed within the shell. The fluid comprising a drug is pressurized through the passage as the pressure member is advanced within the elongated shell. Upon this advancement of the pressure member, the second portion is exfoliated or recoiled from the inner sidewall.

Due to the co-extrusion of the rigid elongated shell and the collapsible cartridge, different materials can be selected to obtain a good grip between the collapsible cartridge and the inner sidewall while at the same time, a good recoilability upon advancement of the pressure member.

For this purpose, the rigid elongated shell may comprise at least a first composite or plastic while the collapsible cartridge may comprise at least a second composite or plastic. The rigid shell as well as the collapsible cartridge may each comprise a respective inner and outer sidewall. In another embodiment, the rigid shell and/or the collapsible cartridge may comprise a multi-layer composite.

In another embodiment, the adhesion between the inner sidewall of the rigid shell and the second portion of the collapsible cartridge may be smaller than an adhesion between two portions of an inner sidewall of the second portion of the collapsible cartridge. The difference in the adhesion may result in “exfoliation” of the material of the second portion of the collapsible cartridge without pushing the second portion along the inner sidewall of the elongated shell. As a result, substantially all fluid within the collapsible cartridge can be pressurized through the passage of the rigid elongated shell.

Further, a static friction between the inner sidewall of the rigid shell and the second portion of the collapsible cartridge may be smaller than a dynamic friction between the inner sidewall of the rigid shell and the second portion. Consequently, the second portion of the collapsible cartridge can be detached from the inner sidewall more easily than pushing the inner portion along the inner sidewall of the rigid shell. As a result, recoilement or exfoliation of the second portion is ensured.

To achieve the difference between the adhesion, the static and dynamic friction as mentioned before, the collapsible cartridge may comprise in an embodiment a first material forming an outer sidewall of the second portion and a second material forming an inner sidewall of the second portion. Particularly, a static friction between the inner sidewall of the rigid shell and the outer sidewall of the second portion may be larger than a static friction between two parts of the inner sidewall of the second portion. It is therefore easier to detach the inner sidewall of the shell from the outer sidewall of the second portion than to detach two parts of the inner sidewall of the second portion of the collapsible cartridge.

In another embodiment, the first portion of the collapsible cartridge is based apart from the inner sidewall of the rigid shell.

To provide guidance for a pressure member slidably disposed within the rigid shell to smoothen any drug delivery, the distal end of the collapsible cartridge may comprise a small recess. The pressure member may be adapted to fit at least partly into that recess as the pressure member is disposed within the rigid elongated shell. As the pressure member is advanced within the rigid shell, the recess guides the pressure member to prevent or at least hinder the pressure member to come into contact with the sidewall of the rigid shell. Consequently, the pressure member may substantially move freely within the rigid shell to increase accuracy of drug delivery.

In a further embodiment, the distal end of the collapsible cartridge may comprise generally a guiding member to guide the pressure member when it is advanced within the rigid shell.

In another embodiment, the pressure member may comprise a truncated cone or frustum, wherein a smaller top surface of the truncated cone or frustum is facing the second portion of the collapsible cartridge. When the pressure member is advanced within the shell, the truncated cone may facilitate recoilement of the second portion of the cartridge. In other words, the second portion of the collapsible cartridge is recoiled from the inner sidewall of the rigid shell under the guidance of the truncated cone of the pressure member.

In a further embodiment, an adhesion or static friction of the material of the pressure member and the material of the collapsible cartridge may be very small compared to the adhesion or friction between the material of the collapsible cartridge and the inner sidewall. Accordingly, the material of the collapsible cartridge may slide quite easily along the pressure member.

In a further embodiment, a diameter of the pressure member is by at least 2 times a wall thickness of the collapsible cartridge smaller than a diameter of the rigid shell. Particularly, the diameter of the pressure member may be at least four times a wall thickness of the collapsible cartridge smaller than the diameter of the rigid shell.

The smaller pressure member allows a substantially free movement of the pressure member along the rigid shell and may prevent the pressure member to come into contact with the rigid shell, which may decrease accuracy of drug delivery. Further, the recoiled material of the collapsible cartridge can be disposed in the space between the pressure member and the inner sidewall of the rigid shell.

In another embodiment, the diameter of the pressure member may be maximal ten times a wall thickness of the collapsible cartridge smaller than the diameter of the rigid shell. As a result, less or even no liquid remains in the recoiled collapsible cartridge as the pressure member advances within the rigid shell.

For manufacturing such drug delivery system, a rigid shell and a collapsible cartridge are simultaneously formed and detachably connected, wherein the rigid shell comprises a sidewall, an opening and a passage, and the collapsible cartridge is disposed within the rigid shell with a first portion being coupled to the passage and a second portion being in contact with a sidewall of the rigid shell. A fluid comprising a drug is provided within the collapsible cartridge.

The method for manufacturing allows a simple, fast, well controlled and cheap way of manufacturing such system. In another embodiment, a pressure member is provided and disposed within the rigid shell such that as the pressure member advances within the shell, a fluid, which comprises a drug and is contained in the collapsible cartridge, is pressurized, while the second portion is recoiled from the inner sidewall.

### BRIEF DESCRIPTION OF THE FIGURES

Other features will become apparent from the following detailed description when considered in conjunction with the accompanying drawings. In the drawings:

FIG. 1 shows a cross-section of an embodiment of a drug delivery system according to the present invention,

FIG. 2 schematically illustrates a cross-section including a cut through the collapsible cartridge of a drug delivery system according to the present invention,

FIG. 3 shows a schematic view of a portion of the drug delivery system according to another embodiment of the present invention,

FIG. 4 shows yet another cross-section of portion of an embodiment according to the present invention,

FIG. 5 illustrates a schematic view in different planes of yet another embodiment of the invention.

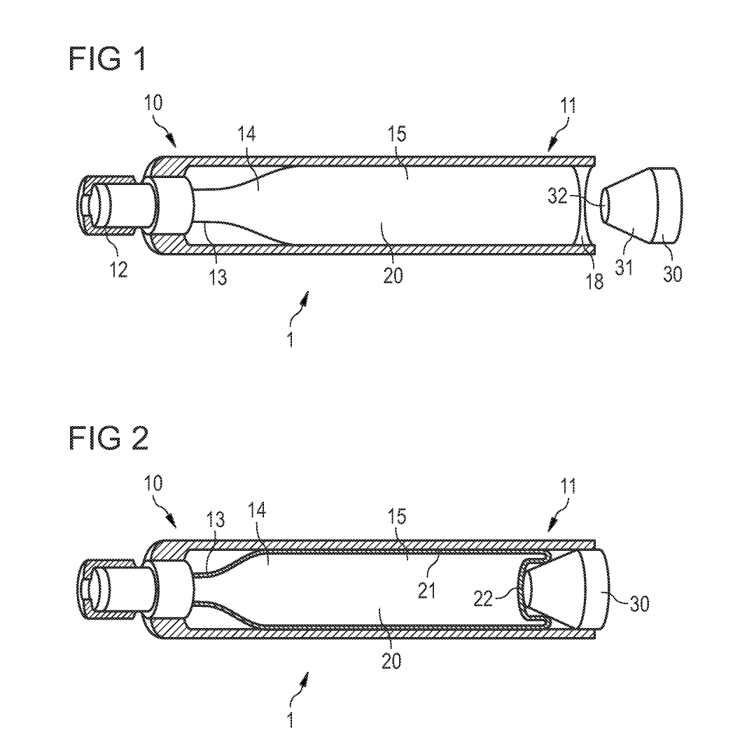

It should be noted that the description of the drug delivery system as shown in the following figures is merely illustrative. Portions or parts of the drug delivery system are illustrated enlarged with respect to other parts. However, the dimensions of portions and parts of the drug delivery system are for illustrational purposes only and do not represent real dimensions or ratios. Similar parts may comprise the same reference. In addition, the different aspects disclosed and explained in greater detail with respect to one of the embodiments can be incorporated into a different embodiment shown herein. Any combination featuring aspects from different embodiments can be incorporated in a drug delivery device.

### DETAILED DESCRIPTION

The term drug or medication, as used herein, preferably means a pharmaceutical formulation containing at least one pharmaceutically active compound,
wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(w-carboxyheptadecanoyl)-des(B30) human insulin and B29-N-(w-carboxyheptadecanoyl) human insulin.

Exendin-4 for example means Exendin-4(1-39), a peptide of the sequence H-His-Gly-Glu-Gly-Thr-Phe-Thr-S er-Asp-Leu-S er-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2 .

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 [Asp28] Exendin-4(1-39),
des Pro36 [IsoAsp28] Exendin-4(1-39),
des Pro36 [Met(0)14, Asp28] Exendin-4(1-39),
des Pro36 [Met(0)14, IsoAsp28] Exendin-4(1-39),
des Pro36 [Trp(02)25, Asp28] Exendin-4(1-39),
des Pro36 [Trp(02)25, IsoAsp28] Exendin-4(1-39),
des Pro36 [Met(0)14 Trp(02)25, Asp28] Exendin-4(1-39),
des Pro36 [Met(0)14 Trp(02)25, IsoAsp28] Exendin-4(1-39); or
des Pro36 [Asp28] Exendin-4(1-39),
des Pro36 [IsoAsp28] Exendin-4(1-39),
des Pro36 [Met(0)14, Asp28] Exendin-4(1-39),
des Pro36 [Met(0)14, IsoAsp28] Exendin-4(1-39),
des Pro36 [Trp(02)25, Asp28] Exendin-4(1-39),
des Pro36 [Trp(02)25, IsoAsp28] Exendin-4(1-39),
des Pro36 [Met(0)14 Trp(02)25, Asp28] Exendin-4(1-39),
des Pro36 [Met(0)14 Trp(02)25, IsoAsp28] Exendin-4(1-39),
wherein the group -Lys6-NH2 may be bound to the C-terminus of the Exendin-4 derivative;
or an Exendin-4 derivative of the sequence
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(02)25, Asp28] Exendin-4(1-39)-Lys6-NH2,
H-des Asp28 Pro36, Pro37, Pro38 [Met(O)14, Trp(02)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(02)25, Asp28] Exendin-4(1-39)-NH2,
des Pro36, Pro37, Pro38 [Met(O)14, Trp(02)25, Asp28] Exendin-4(1-39)-(Lys)6-NH2,
H-(Lys)6-des Pro36, Pro37, Pro38 [Met(O)14, Trp(02)25, Asp28] Exendin-4(S1-39)-(Lys)6-NH2,
H-Asn-(Glu)5-des Pro36, Pro37, Pro38 [Met(O)14, Trp(02)25, Asp28] Exendin-4(1-39)-(Lys)6-NH2;
or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

The drug delivery system 1 may be configured to deliver a plurality of fixed or user-settable doses of a drug. The drug delivery system 1 may be a pen-type device, preferably a pen-type injector. It comprises a rigid shell 10 with an opening 18 on the one end and a passage 12 on the other, to which a needle unit can be attached. The rigid shell 10 has the form of a cylinder with its proximal end close to passage 12 and its distal end 11 close to opening 18. Passage 12 goes through the bottom plate of the rigid shell 10 and comprises a second portion 13 within the shell. Said second portion of the passage 12 is coupled to a collectable cartridge 20 comprising the form of a bottle with a first portion connected to passage part 13 and a second portion 15 being in contact with the sidewall of the rigid shell 10.

The collapsible cartridge has a length, which is slightly smaller than the respective length of the rigid shell 10, such that a small part at the distal end 11 near the opening 18 of the shell 10 is left free. This will allow pressure member 30 to be disposed in the rigid elongated shell 10 to recoil or exfoliate the second portion of collapsible cartridge 20.

Pressure member 30 comprises a truncated cone with sidewall 31 and top surface 32. The top surface 32 is facing the bottom 22 of the collapsible cartridge upon disposal of pressure member 30 within the rigid shell 10. As the pressure member 30 advances within the rigid shell, second portion 15 of the collapsible cartridge is exfoliated from inner sidewall of shell 10, thereby decreasing the volume of cartridge 20 and urging the fluid within the cartridge through passage 12 and 13.

FIG. 2 shows a respective embodiment, in which pressure member 30 is disposed within rigid elongated shell 10. Collapsible cartridge 20 is cut open so that inner parts of cartridge 20 can be seen more clearly. The bottom 22 of collapsible cartridge 20 is in touch with top surface 32 of pressure member 30. As pressure member 30 advances within the shell, the material of second portion 15 of cartridge 20 detaches and exfoliate from the inner sidewall of the rigid shell and coils up to be in contact with pressure member 30.

For this purpose, the adhesion or friction between the outer sidewall of second portion 15 of collapsible cartridge 20 and the inner sidewall of rigid shell 10 is smaller than the adhesion between two parts or portions of inner sidewall 21 of second portion 15 of collapsible cartridge 20. As a result, second portion of collapsible cartridge 20 recoils from inner sidewall of shell 10 and is not pushed forward within the shell due to the difference in adhesion. At the same time, the second portion 15 slides along the truncated cone of pressure member 30 and is disposed between the inner sidewall of rigid shell 10 and pressure member 30 such that substantially all liquid in the space between the material of second portion 15 is pressed out. In other words, substantially no fluid remains in the space between the materials of second portion 15 as pressure member 30 advances along the rigid shell.

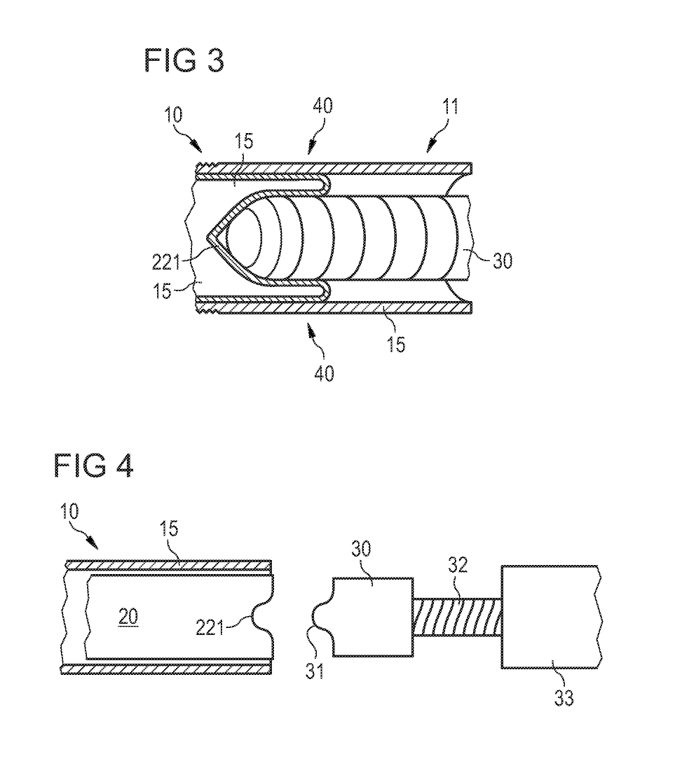

FIG. 3 shows an embodiment which illustrates the disposal of material of second portion 15 of collapsible cartridge 20 between pressure member 30 and rigid shell 10. In area 40, the material of the collapsible cartridge 20 is foiled such that some portion is in contact with the inner sidewall of rigid shell 10 while other portions of material of collapsible cartridge 20 within second portion 15 are in contact with pressure member 30.

Collapsible cartridge 20 comprises a recess 221 at its bottom. That recess is used as a guiding member to guide pressure member 30 as it advances along rigid shell 10. This may allow a smoother and accurate delivery of fluid as pressure member 30 advances along the rigid shell. Diameter of pressure member 30 is chosen to be at least four times smaller than diameter of rigid shell 10 as indicated in FIG. 3. This allows disposing material of second portion 15 of collapsible cartridge 20 in the free space between pressure member 30 and inner sidewall of rigid shell 10. At the same time, fluid is pressed out from the now overlapping material of second portion 15 in area 40.

The collapsible cartridge according to the embodiment of FIG. 3 may comprise a multi-layer composite in which the adhesion between material of the inner sidewall of second portion 15 of collapsible cartridge 20 may be smaller than the respective adhesion between the outer sidewall of second portion 15 and the inner sidewall of rigid shell 10 and/or the adhesion between the outer sidewall of second portion 15 and the sidewall of pressure member 30. As a result, second portion 15 is recoiled from the inner sidewall as pressure member 30 advances within the rigid shell 10 and not pushed forward along the rigid shell.

FIG. 4 shows another embodiment of a fluid drug delivery system according to the present invention. In this embodiment, collapsible cartridge 20 is flash-mounted or disposed within rigid shell 10 such that the bottom with recess 221 of collapsible cartridge 20 ends at the opening of rigid shell 10. Recess 221 acts as a guiding member for pressure member 30 comprising with enhancement 31 mounted on top of pressure member 30. Enhancement 31 fits into recess 221 to guide pressure member 30 along the rigid shell without contacting the inner sidewall. Pressure member 30 is in contact with piston rod 34, that piston rod 34 being part of an injector dose device 33. The fluid drug delivery system according to the present invention is placed in such dose delivery device, which can be a pen-injector type used by a patient to deliver an accurate amount of a drug fluid.

The smaller diameter of pressure member 30 reduces any adhesion between pressure member 30 and the rigid shell. The material used for collapsible cartridge 20 and inner sidewall of rigid shell 10 results in a specific adhesion between second portion 15 of collapsible cartridge 20 and the inner sidewall of rigid shell 10, such that second portion 15 is recoiled from the inner sidewall due to an advancement of pressure member 30. This adhesion also results in additional volume stability of cartridge 20. Accordingly, an additional valve or outlet between passage 12 and an injection needle attached thereto is not required.

The space between pressure member 30 and the inner sidewall of rigid shell 10 is chosen to be large enough that second portion 15 of collapsible cartridge 20 is recoiled precisely without being pushed together but small enough that any liquid in this area is substantially pressed out.

The fluid drug delivery system can be manufactured using co-extrusion blow process, forming rigid shell 10 and flexible collapsible cartridge substantial simultaneously. For this purpose, collapsible cartridge as well as rigid shell may comprise different kinds of plastics or composites and even multi-layer composites. The materials used for rigid shell 10 and collapsible cartridge 20 may be transparent or at least semi-transparent or transparent in some areas to optically control the remaining volume of fluid within the cartridge.

In another embodiment, the flexible and compressible cartridge is arranged within the rigid shell after forming the rigid shell without forming any cavity between the cartridge and inner sidewall of the rigid shell. To prevent a folding of collapsible cartridge and thereby generating locally small fluid chambers, a respective adhesion between collapsible cartridge 20 and the rigid shell must be generated. Still, this adhesion must be small enough to recoil second portion of collapsible cartridge 20 from the inner sidewall.

Rigid shell 10 may comprise the form of an ampoule or a pharmaceutical file.

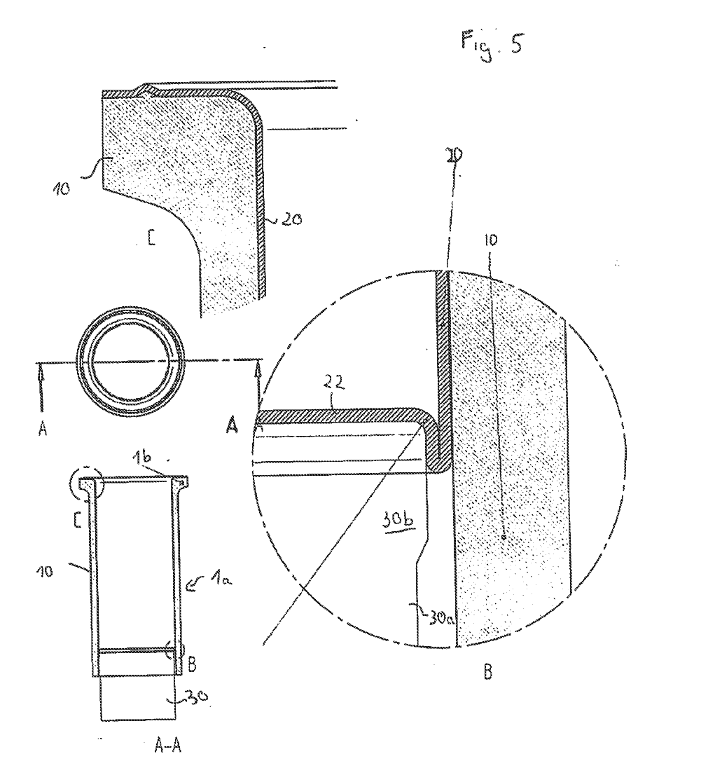

FIG. 5 illustrates a schematic view of another embodiment of the present invention. The container shown herein is a replaceable part of a pen-type injector and can be arranged in the drug delivery system. The container la comprises a hard outer shell 10 and a flexible and compressible inner cartridge 20 or bag. The rigid shell 10 has no bottom at its distal end, but an opening through which pressure member 30 or plung of the injector can be inserted. Pressure member 30 will contact the flexible bag after it has been inserted into the injector device.

Rigid shell 10 as well as collapsible cartridge 20 may be co-extruded in one or more common manufacturing steps or formed in a 2K-injection molding process, wherein collapsible cartridge 20 is injected into rigid shell 10. Accordingly, the outer surface of the collapsible cartridge is equal or slightly smaller than the inner surface of rigid shell 10 in case of a two step manufacturing process or approximately equal to the inner surface in case of a co-extrusion or injecting process.

As illustrated in FIG. 5, container 1 comprises a double-walled ampoule with a rigid outer shell and a compressible and flexible inner catridge. Container 1 is replaceable and arranged in the injector to form a drug delivery device. The flexible inner cartridge 20 comprises approximately the same length as the outer shell 10.

After inserting the filled container into the injection device, the pressure member is inserted from behind into the rigid shell 10, but without being in contact with the outer rigid shell 10. For this purpose the pressure member 30 may comprise two different diameter, both smaller than an inner diameter of the rigid shell 10. A first portion 30b of pressure member 30 adjacent to bottom 22 of collapsible catridge 20 comprises a diameter approximately equal to the inner diameter of the rigid shell less four times the thickness of the collapsible cartridge's sidewall. A second portion 30a may have the same or an even smaller diameter, such that pressure member 30 avoids contact with the rigid shell's sidewall. The length of portion 30b may be at least half the length of flexible inner cartridge 20, so that no space for the fluid contained in the cartridge is provided between pressure member 30 and the inner side wall of rigid shell 10 when the collapsible cartridge is partially or fully collapsed. Pressure member 30 is guided only by feed-forward mechanism in the injector. As the feed-forward mechanism is integrated in to the pen or injector itself, it provides an appropriate gear (eg, spindle-nut drive) and and a high dose accuracy, respectively.

When the pressure member is moved forward, collapsible cartridge is recoiled as illustrated. Its internal pressure will prevent local wells being separated during drug dispension. The container 1a does not comprise a holder for the needle, as the needle and its holder are part of the injector in this embodiment. For the purpose of drug delivery, a septum at the proximal end of container 1a will be penetrated by a portion of the needle after the container is inserted into the injector. In the embodiment of FIG. 5, the septum of the collapsible cartridge is arranged adjacent to protrusion lb of rigid shell 10. Hence, even a shoulder area of the container is not absolutely necessary for a 2K-production of the container as illustrated.

Since the injector can be reused including the piston and the pressure member, the collapsible catridge is not attached to the pressure member. The pressure member can thereby have the same shape as the center in the collapsible cartridge in order to ensure the exact contact fit of both partners and guarantee steady stripping of the entire length guaranteed. To reduce the residual volume of the drug, the proximal end of the collapsible cartridge and/or the rigid shell may comprise also a conical shape.

1. A fluid drug delivery system, comprising:
a rigid shell having an inner sidewall, an opening and a passage;
a collapsible cartridge with a distal end and a proximal end, the collapsible cartridge being arranged within the rigid shell with its distal end being closer to the opening than the proximal end and further having a first portion including the proximal end and a second portion including the distal end, wherein the first portion is coupled to the passage and the second portion is in contact with the inner sidewall of the rigid shell,
wherein the second portion can be exfoliated from the inner sidewall to urge a fluid, which comprises a drug and is contained in the collapsible cartridge, through the passage,
wherein the rigid shell and the collapsible cartridge comprise approximately the same length.

2. The drug delivery system according to claim 1, wherein the rigid shell comprises at least a first composite or plastic, and the collapsible cartridge comprises at least a second composite or plastic.

3. The drug delivery system according to claim 1, wherein the rigid shell and/or the collapsible cartridge comprise a multi-layer composite.

4. The drug delivery system according to claim 1, wherein an adhesion between the inner sidewall of the rigid shell and the second portion of the collapsible cartridge is larger than an adhesion between two portions of an inner sidewall of the second portion of the collapsible cartridge.

5. The drug delivery system according to claim 1, wherein a static friction between the inner sidewall of the rigid shell and the second portion is smaller than a dynamic friction between the inner sidewall of the rigid shell and the second portion.

6. The drug delivery system according to claim 1, wherein the collapsible cartridge comprises a first material forming an outer sidewall of the second portion and a second material forming an inner sidewall of the second portion.

7. The drug delivery system according to claim 6, wherein a static friction between the inner sidewall of the rigid shell and the outer sidewall of the second portion is larger than a static friction between two parts of the inner sidewall of the second portion.

8. The drug delivery system according to claim 1, wherein the first portion is spaced apart from the inner sidewall of the rigid shell.

9. The drug delivery system according claim 1, wherein the distal end of the collapsible cartridge comprises a small recess.

10. The drug delivery system according to claim 1, comprising a pressure member which is adapted to be slidably disposed within the rigid shell, wherein the second portion of the collapsible cartridge is recoiled from the inner sidewall when the pressure member is advanced within the rigid shell.

11. The drug delivery system according to claim 10, wherein the pressure member is adapted to fit at least partly into said recess as the pressure member is disposed within the rigid shell.

12. The drug delivery system according to claim 10, wherein the distal end of the collapsible cartridge comprises a guiding member to guide the pressure member as it is advanced within the shell.

13. The drug delivery system according to claim 10, wherein the pressure member comprises a truncated cone or frustum, wherein a smaller top surface of the truncated cone or frustum is facing the second portion of the collapsible cartridge.

14. The drug delivery system according to claim 10, wherein a diameter of the pressure member is by at least 2 times a wall thickness of the collapsible cartridge smaller than a diameter of the rigid shell.

15. The drug delivery system according to claim 1, wherein the compressible cartridge and rigid shell are co-extruded.

16. The drug delivery system according to claim 1, wherein the pressure member is guided contactless in the rigid shell with respect to the sidewall of the rigid shell.

17. The drug delivery device according to claim 1, wherein the collapsible cartridge comprises a septum at its proximal end, said septum being adapted to be penetrated by a needle for drug delivering.

18. Method for manufacturing a drug delivery system, comprising:
simultaneously forming and detachably connecting a rigid shell, which has a sidewall, an opening and a passage, and a collapsible cartridge within the rigid shell, wherein the collapsible cartridge comprises a first portion being coupled to the passage and a second portion being in contact with the sidewall of the rigid shell;
providing a fluid which comprises a drug within the collapsible cartridge.
